(19) United States
(12) Patent Application Publication
(10) Pub. No.: US 2006/0024333 A1
(43) Pub. Date: Feb. 2, 2006

(54) Use of parasitic biological agents for prevention and control of allergic and other IgE-mediated disorders

(76) Inventor: Daniel J. O'Connor, Pennington, NJ (US)

Correspondence Address: COLLIER SHANNON SCOTT, PLLC, 3050 K STREET, NW, SUITE 400, WASHINGTON, DC, 20007, US

(21) Appl. No.: 11/187,819

(22) Filed: Jul. 25, 2005

Related U.S. Application Data
(60) Provisional application No. 60/590,905, filed on Jul. 26, 2004.
(60) Provisional application No. 60/647,032, filed on Jan. 27, 2005.

(51) Int. Cl. A61K 39/00; A01K 67/033

(52) U.S. Cl. 424/265.1; 800/13

(57) ABSTRACT

The present invention describes using, on a repetitive basis, a non-human colonizing helminth compound, in an amount sufficient to establish as needed a transitory helminth infection and or to simulate in a parasitic helminth infection with helminth excretory and secretory products (ESP), thereby having immunosuppressive effect against benign antigens and or stimulating a regulatory immune response characterized by the production of T helper cells 2 (Th2), T regulatory helper cells (TReg) and certain cytokines, including, but not limited to interleukin 10 (IL-10), as a therapy or prophylaxis of allergy and other IgE-mediated disorders, which are marked by an inappropriate immunoglobulin E (IgE) immune response including, but not limited to an abnormal IgE antibody production to benign antigens. The invention presents using helminth compound by administering it in a frequency and amount sufficient to eliminate or ameliorate the inappropriate immune response in an asthmatic and or allergic individual. This invention is generally directed to diseases IgE antibody-mediated disorders, including asthma, allergies, hypersensitivity and anaphylactic reactions. More specifically, the present invention is directed toward the treatment of certain common food allergies, such as an allergy to peanut, tree nut, milk, egg, wheat, and shellfish (CFA's).

### CROSS REFERENCE TO RELATED APPLICATIONS

The present invention relates to, and is entitled to the benefit of the earlier filing date and priority of, Application Nos. 60/590,905, filed on Jul. 26, 2004 and 60/647,032, filed on Jan. 27, 2005, both of which are herein incorporated by reference as if fully set forth.

### FIELD OF THE INVENTION

The present invention relates to compositions and methods of treating disease states that are marked by abnormal IgE immune responses to benign antigens, including an inappropriate Th2 cell production. Specifically, the invention relates to administration of helminth compounds to reduce the excessive IgE immune response in a human. The methods and compositions of the invention may be used to treat allergies, including but not limited to food allergies, allergies caused by pollen, pet dander, dust mites as well as asthma.

### BACKGROUND OF THE INVENTION

Helminths are elaborate multicellular worms with complex life cycles and development. Helminths inhabit their host's gastrointestinal (GI) tract and, in order to survive, establish a relationship with the host's mucosal defenses. The nematodes (nonsegmented roundworms) and the platyhelminths (flatworms) are the two groups of helminths that inhabit the human intestines. Helminth infections are highly prevalent in human populations, particularly in tropical and subtropical countries. Helminths that are classified as parasites that infect humans are so classified because they are known to have a pathological effect on the human host. Helminths with no associated pathology in the host are known as comiciles. Twenty-six species of helminth parasites have been reported to infect humans. Among these parasitical helminths, nematode species that colonize the GI tract are of concern in terms of overall morbidity. The four most prevalent species of nematodes—Ascaris lumbricoides, Trichuris trichiura, Necator americanus and Ancylostoma duodenale—infect more than a billion people worldwide (M. Chan, Parasite Today, 13: 438-443, 1997). The prevalence of parasitical helminths is highest in rural and underdeveloped areas characterized by overcrowding, poor sanitation and an impure food and/or water supply. When compared to areas where the standard of living is higher, asthma and allergies occur at a much lower rate in these rural and underdeveloped regions (Lancet 351, 1225 (1998)).

Allergy and asthma are diseases marked by inappropriate immune system responses to benign, allergy causing substances, such as pet dander, dust mites and pollen. Allergy and asthma can cause airways to become blocked or narrowed. Generally, they cause a shortness of breath, wheezing, coughing, breathing trouble and other symptoms. If an asthma or allergic attack is severe, the person may need emergency treatment to restore normal breathing. In certain cases, this inappropriate immune system response is so overwhelming that it can be fatal.

In the United States there are more than 50 million people, approximately twenty percent of the population, who suffer from the allergic diseases of asthma and/or allergies (Safety & Health Policy Center, A Division of the National Safety Council, 1025 Connecticut Avenue, NW, Suite 1200, Washington, D.C. 20036. Apr. 8, 2004). Approximately 11.4 million Americans, or 4% of the population, are allergic to certain foods, such as, but not limited to, peanuts, milk, eggs, wheat, shellfish, and others (or common food allergens (CFA's)). Peanut allergy is one of the most severe food allergies due to its persistency and life-threatening character (H. Sampson, et al., J. Pediatr., 1985;107: 669-675). The prevalence of peanut allergy in the Western world has been estimated at from 1 in 10,000 up to 1 in 200 and seems to be increasing during the last decade (S. Tariq, et al., Br. Med. J., 1996; 313: 514 517). The number of Americans who have food allergies has risen over the past ten years, when scientists believed that less than 1% of the population were affected by food allergies. The incidence of food allergy continues to increase around the world, and has become a food safety and public health concern of governments, health care professionals, the food industry and schools (Food Allergy News, Volume 14, No. 3, February, 2005, a publication of The Food Allergy & Anaphylaxis Network). Americans spend at least $5 billion a year in an attempt to cope with these afflictions.

In the United States, asthma is the most common chronic disease of childhood. The number of young people and children with asthma is rising. About 17 million Americans have asthma and almost 9 million children have been diagnosed with asthma at some point in their life. Nearly one in 13 school-aged children has asthma. Between 1980-1994, asthma among children under five years old increased by 160 percent. Nearly one in five of all pediatric emergency room visits is asthma-related. This is an increase of approximately 45 percent in the past decade. Asthma attacks in children contribute to parents making nearly a million emergency room visits every year, which accounts for half of the $2 billion cost of treating children with the illness.

In the United States, asthma causes approximately 5,000 deaths per year. Peanut allergies account for 50 to 100 deaths in the United States each year (CNN, Education, Friday, Jan. 30, 2004). Although most asthmatics that die of the disease are more than 50 years old, rates of asthma death have increased in almost all age groups. Most asthma deaths occur in urban areas. Worldwide, the prevalence of asthma has increased and continues to increase dramatically (R. Beasley, Global Burden of Asthma. (Commissioned by Global Initiate for Asthma (GINA). Data obtained on the burden of asthma in 20 different regions worldwide from literature primarily published through the International Study of Asthma and Allergies in Childhood (ISAAC) and the European Community Respiratory Health Survey (ECHRS). The Global Initiative for Asthma (GINA) program was initiated with the U.S. National Heart, Lung, and Blood Institute, NIH and the World Health Organization (WHO) in an effort to raise awareness among public health and government officials, health care workers, and the general public that asthma was on the increase)). The international patterns of asthma prevalence are not explained by the current knowledge of the causation of asthma. Asthma has become more common in both children and adults around the world in recent decades. The increase in the prevalence of asthma has been associated with an increase in atopic sensitization, and is paralleled by similar increases in other allergic disorders such as eczema and rhinitis.

It is also estimated that worldwide as many as 300 million people of all ages, and all ethnic backgrounds, suffer from asthma and the burden of this disease to governments, health care systems, families, and patients is increasing worldwide. Asthma is one of the most common chronic diseases in the world. With the projected increase in the proportion of the world's population dwelling in urban areas from 45% to 59% by 2025, there is likely to be a marked increase in the number of asthmatics worldwide over the next two decades. Some sources estimate that there may be an additional 100 million persons with asthma by 2025. This trend has been building for decades. In fact, there has been a significant global increase in the prevalence of allergic diseases over the past 40 years (Ibid.).

There is a considerably lower prevalence of allergic diseases in developing countries (Lancet 351, 1225 (1998)). There are also clear differences in the prevalence of allergies between rural and urban areas within one country. For example, in Ethiopia, asthma is more prevalent in urban areas than in rural villages (H. Yemaneberhan et al., Lancet 350, 85 (1997)), and asthma is more common in residents of urban Germany than in farmers living in rural Bavaria (0. S. von Ehrenstein et al., Clin. Exp. Allergy 30, 187 (2000)).

Because allergic diseases are most prevalent in highly industrialized regions and the relative absence of allergic disease in underdeveloped regions, it has been suggested that there is some critical environmental factor responsible for the increasing frequency of asthma and allergies in regions as they develop. People in industrialized regions live in increasingly hygienic environments and, as a result, acquire helminths much less frequently than those people living in rural areas. The increase of asthma and allergic diseases in the industrialized world has also been explained by a decline in bacterial and viral infections during childhood. This explanation, commonly known as the “Hygiene Hypothesis,” is explained from an immunological perspective by theorizing that bacterial and viral infections during early life (direct the maturing immune system toward the functional T-helper 1 cells (Th1)), which counterbalance pro-allergic responses of T-helper 2 cells (Th2) (D. P. Strachan., Br. Med. J. 299:1295 (1989)). Thus, it is thought that an overall reduction in bacterial and viral infections during childhood result in a weak Th1 development and unrestrained Th2 responses that allow an increase in allergy (W. Cookson,. and M. Moffatt, Science, 275:41(1997)).

The Hygiene Hypothesis is contradicted by observations that the prevalence of Th1-autoimmune diseases, such as Crohn's disease, are also increasing and that Th2-skewed helminth infections are disassociated with allergy and asthma. Further, this theory fails to consider that the worldwide trend toward greater hygiene (i.e., food and water purification, increased sanitation efforts and medicines to treat parasitical helminth infections) is resulting in rendering humans free of parasitical helminths. Stated another way, the elimination of the chronic immune system stimulation induced by a helminthic may account for the increase in asthma and allergies.

The potential connection between asthma, allergies and parasites is not new. In the 1960's and 1970's there was vigorous scientific debate around the idea that helminths provide a protective effect against allergies. In addition to anecdotal reports of protection from hay fever by ingestion of Ascaris spp. (roundworm) eggs (J. A. Turton, Lancet 2, 686 (1976)), a meta-analysis of data from early surveys showed that, despite the variation in methodology and clinical assessment of allergies, the prevalence of parasitic helminth infections was negatively associated with the prevalence of asthma (S. Masters, et al., Epidemiol. Rev. 7, 49 (1985)).

Humans, as parasitic helminth hosts, can acquire various parasitic helminth species through contact with soil, food or water contaminated with the infective form of the parasite. Children in rural areas of the world most frequently harbor parasitic helminth infections because of their close contact with soil and less than optimal hygienic practices. When a parasitic helminth enters the body, the body's immune mechanism is activated and the immune response causes the production of billions of Y-shaped antibodies to the foreign proteins shed by helminth.

Helminths inhabit the host's GI tract and, in order to survive, establish a relationship with the host's mucosal defenses. The GI tract is an organ for digestion, absorption and excretion. It is one of the largest immunological organs of the body, and it serves as the first line of defense against intestinal pathogens (e.g., bacteria, parasites). Gut-associated lymphoid tissues (GALT) make up approximately 25% by weight of the gut mucosa and submucosa and thus constitute the largest extrathymic site of lymphocytes in humans (M. McBurney, Can. J. Physiol. Pharmacol. 1993;72:260-265). Cells in GALT respond to intestinal pathogens by processing antigens for recognition by lymphocytes, by initiating a cascade of specialized immune responses to the antigens, by regulating the migration of immune mediators from the periphery to the infected gut and by participating directly in cytotoxic activities that limit parasite establishment and survival. In addition to these specific immunological responses, the GI tract performs nonspecific barrier functions (R. Van der Hulst, et al., Nutrition 1998;14:1-6), (F. Welsh, et al., Gut 1998;42:396-401). Mucus secretion and formation of tight cell junctions prevent the entry of bacteria and other pathogenic antigens, and rapid mucosal turnover enables the repair of epithelial or lymphoid cells damaged by parasitic infections.

The gut mucosal immune system consists of two anatomically and functionally distinct compartments: (1) the specialized local inductive sites (Peyer's patches, isolated lymphoid follicles, mesenteric lymph nodes), where intestinal antigens are first recognized; and (2) diffuse effector sites (intraepithelium and lamina propria), where the outcome of an effective immune response is elimination of the infectious agent. Luminal antigens are transported across epithelial barriers either by specialized epithelial M cells or by intraepithelial lymphocytes (mostly T cells) to the organized lymphoid tissues within the mucosa (e.g., Peyer's patches). After epithelial transport, antigens are processed and presented by antigen-presenting cells (APC) such as dendritic cells, B cells, macrophages and other intestinal epithelial cells. Naive T lymphocytes first interact with antigen-primed APC in aggregated Peyer's patches and single lymphoid follicles and then further differentiate in the germinal centers of the lymphoid follicles. Thereafter, the antigen-specific T and B cells leave the epithelial barrier to collect in the mesenteric lymph nodes (MLN), which drain the mucosa and supply the peripheral bloodstream with gut-derived or locally activated immune cells, or both.

From the blood, the lymphocytes migrate to systemic lymphoid tissues such as the spleen and peripheral lymph nodes, where the lymphocytes proliferate and mature either into effector lymphocytes, which secrete cytokines and mediate T cell-dependent humoral immunity, or into memory cells that can respond rapidly to the infection on secondary encounter. Peripheral lymphocytes can preferentially leave the blood vessels and move into the intestinal lamina propria and intraepithelium by expressing adhesion receptors that are recognized by specific endothelial molecules lining the gut mucosal lymphoid tissues. As a result, most of the antigen-committed and differentiated lymphocytes that enter the effector sites of GALT are likely to have had prior contact with, and specific activation by, parasite antigens located in the gut mucosa.

The continuous migration of lymphocytes from intestinal lymphoid tissues to the bloodstream and back enables the GALT to carry out two important roles in the defense against intestinal parasites. First, it allows delivery of the parasite antigen to peripheral sites, initiating a widely disseminated immune response, and second, it promotes trafficking of gut-derived lymphocytes from the blood to effector sites within the intestinal epithelium. Gut-associated lymphocytes further contribute to host defense against GI parasites by secreting cytokines that regulate the appropriateness, magnitude and phenotypic expression of immune responses.

Lymphocytes are one of the five kinds of white blood cells or leukocytes. There are several kinds of lymphocytes, each with different functions to perform. The most common types of lymphocytes are B lymphocytes or B cells, which are responsible for making antibodies. B cells are specialized white blood cells produced in the bone marrow. T lymphocytes or T cells, one of which is T helper cells or Th cells, enhance the production of antibodies by B cells. Although bone marrow is the ultimate source of lymphocytes, the lymphocytes that will become T cells migrate from the bone marrow to the thymus where they mature. Both B cells and T cells also take up residence in lymph nodes, the spleen and other tissues where they encounter antigens, continue to divide by mitosis, and mature into fully functional cells. Each B cell contains multiple copies of one kind of antibody as a surface receptor for antigens. The entire population of B cells has the ability to specifically bind to millions of different antigens.

Depending on the type of antigenic stimulus, undifferentiated T helper (Th) cells transform into either Th1 or Th2 cells. Th cells also regulate other cells of the immune system through secretion of molecules called cytokines. Cytokines are messenger substances that regulate the immune system. It is believed that the type of cytokine that is secreted determines the nature of the inflammatory response. In other words, the immune responses usually present as either Th1, which display certain cytokine profiles and which counterbalance pro-allergic responses of Th2, which also display polarized cytokine profiles. For example, the excretion of Th1 causes an inflammatory reaction, while the excretion of Th2 brings about an inflammation-inhibiting reaction in the immune system (There are two distinct kinds of T-helper cells, Th1 and Th2. Th1 participates in cell-mediated immunity. They are essential for controlling such intracellular pathogens as viruses and certain bacteria. Th2 provide help for B cells and, in so doing, are essential for antibody-mediated immunity).

Bacterial, viral and protozoan infections usually stimulate a Th1 response, characterized by elevated levels of Th1 cytokines (i.e., interleukin (IL)-2, IL-12, interferon (IFN)) and effectors such as macrophages, natural killer cells and neutrophils). In such Th1 responses, cell-mediated immunity involving phagocytosis is responsible for the functional immunity. Th1-type inflammations produce large amounts of IFN-y and tumor necrosis factor (TNF)-alpha. In contrast, the immune response to a parasitic helminth depends on the production of Th2 cytokines (e.g., IL-4, IL-5, IL-10, and IL-13), which mediate antibody-dependent effector responses described below. These Th2 cytokines released in the GALT attract progenitors of B cells, mucosal mast cells (MMC) and eosinophils by chemotaxis to the mucosal epithelium, where they proliferate and mature in response to the stimulatory signals of cytokines and parasite and or helminth antigens.

Weinstock et al. (U.S. Pat. No. 6,764,838) discloses the administration of helminthic parasite preparations to treat excessive immune responses in an individual that results in autoimmune disease. Unlike allergies that result from an excessive Th2 response, autoimmune diseases are believed to result from an excessive Th1 response.

Antibodies are needed to control extracellular pathogens, such as parasitic helminths, which are exposed to antibodies in blood, other body fluids and the GI tract. There are five different types of antibodies found in humans. The type of antibody released to combat a parasitic helminth infection, immunoglobulin E, or IgE, is least common of the five. When released, the IgE antibody attaches the lower portion of its Y-shape onto the surface of mucosal mast cells (MMC). MMC, each of which contain thousands of histamine packed globular granules, are found in high concentrations in human skin, in the membranes of the eyes, nose, and throat, and in the lining of the lungs and gut. Each MMC has hundreds of thousands of Y-shaped antibodies protruding from its surface. When a protein shed by a parasitic helminth sticks (or cross-links) between the arms of two adjacent IgE antibodies, a chain reaction occurs that ends with the MMC releasing its thousands of globular granules. The granules, in turn, release their stored histamines, along with other chemicals, which infiltrate the skin and other tissues close to the activated MMC. These chemicals cause all of the symptoms of inflammation, namely itching, dilated and leaky blood vessels, swelling and excess mucus secretion, all of which are effective in isolating and eliminating the parasite before it can multiply. This immune response protects the body.

Although parasitic helminths induce a polarized Th2 response, as described above, they have been shown to confer protections against allergies and asthma. Recent studies have reevaluated findings in South America and Africa using a combination of parameters to assess allergy with careful parasitological diagnosis and have shown a consistent inverse relation between helminth infections (schistosomiasis and intestinal helminths) and either skin reactivity to environmental allergens or clinical scores, such as airway hyper-responsiveness, wheeze, and asthma (S. Masters, Epidemiol. Rev. 7, 49(1985)). In most of these studies, 30% of the studied subjects carried substantial levels of IgE to house dust mite (HDM-IgE); these values correspond to those seen in many industrialized countries. In high-income countries, allergen-specific IgE leads to skin reactivity to mite, but in less developed countries, the presence of specific IgE does not always translate into equivalent numbers of atopic (skin) reactions. In Gabon, only 11% of the school children reacted to mite in a skin prick test (SPT), whereas 32% were positive for HDM-IgE (A. van den Biggelaar et al., Lancet 356, 1723(2000).20; O. A. Nyan et al., Clin. Exp. Allergy 31, 1672 (2001)). High levels of IgE and SPT positivity in affluent societies in central Europe (33%) (J. Riedler et al., Lancet 358, 1129 (2001)) and Australia (32.5%) (A. Faniran, et al., Thorax 54,606 (1999)) are associated with high prevalences of airway disease (12% asthma in central Europe and 21.9% wheeze in Australia).

By contrast, in many low-income countries, such as Gambia (O. A. Nyan et al., Clin. Exp. Allergy 31, 1672 (2001)) and Nigeria (A. Faniran, et al., Thorax 54,606 (1999)), 35.3% and 28.2% atopic reactions translated into only 3.6% asthma and 6% wheeze, respectively. Thus, despite IgE sensitization to environmental allergens, helminth-infested subjects seem to be protected from MMC degranulation.

Researchers found that Ethiopians who showed signs of having had hookworm infestation were far less likely to report asthmatic symptoms. In a study conducted at the University of Nottingham and Jimma University in Ethiopia (Am J Respir Crit Care Med. 2003 May 15;167(10):1369-73), researchers examined over 200 Ethiopians with asthma, and compared them with almost 400 non-asthmatic Ethiopians. Fecal samples were examined for signs of parasitic infection. In total, hookworm was present in 24% of those tested and people with hookworm infestation were only half as likely to have asthmatic symptoms and there was a relationship between the level of hookworm infestation and the prevalence of asthma. The study concluded that there was an increased risk of asthma in urban areas covered by the project partly due to the protective effect of hookworm infestation.

The amount of and chronic nature of helminth infections may be an important variable that may determine whether helminths act as a risk factor for, or confer protection against, allergic diseases. In Venezuela, the classification of helminth-infested populations into those with none, light, or heavy worm burdens shows that light helminth infections are associated with the amplification of allergen-specific IgE responses and a high skin reactivity, whereas heavily parasitized subjects are protected from atopic skin reactivity despite a high degree of sensitization to mite (S. Masters, Epidemiol. Rev. 7, 49 (1985)).

As stated above, a Th2-type response of inflammation commonly seen in allergic reactions is the same Th2 immune response that the body mounts against parasitic helminths. In other words, because external elements, such as dust mites, pollen and peanuts, are inappropriately determined by the immune system of allergic people to be allergens, they are met with the same IgE immune response that the body mounts against a parasitic helminth infection.

When the human body first encounters an allergen like ragweed pollen, the pollen's foreign proteins activate the antibody-dependant effector responses, i.e., the Th2 branch of the immune system, and IgE antibodies are quickly posted on MMC in vulnerable pollen-exposed areas, such as the nose, respiratory tract, and eyes. When the body encounters ragweed proteins again, the IgE antibodies on the MMC catch the foreign proteins and release histamines, which cause inflammation, i.e., a runny nose, sneezing, coughing, and itchy, watery eyes. Similarly, if the foreign proteins are dust mites that find their way to the lungs, the allergic reaction can trigger the wheezing and shortness of breath associated with asthma. In the same way, a meal of shellfish can produce the upset stomach and diarrhea of food allergy.

Allergists do not know why the human immune system attacks such benign substances as pet dander, dust mites and pollen, reacting as if they were parasites. However, allergists do know that IgE-mediated disorders, including asthma, food allergies, hypersensitivity and anaphylactic reactions are unlike any other immune reaction, except for one: the immune system's response to parasites. The difference between a parasite and ragweed, dust mites or peanuts, is that parasitical helminths have an associate pathology if the IgE antibodies do not sufficiently repel them. However, allergens such as ragweed, dust mites or peanuts in an individual without allergic disease are harmless.

The mechanisms by which immune responses to nonpathogenic environmental antigens lead to either allergy or nonharmful immunity are unknown. It has been theorized that the immune responses in healthy and allergic individuals are characterized by a fine balance between allergen-specific T Regulatory 1 cells (TReg) and Th2 cells (J Exp Med. 2004 Jun. 7; 199(11):1567-75. Epub 2004 Jun 01). Single allergen-specific T cells constitute a very small fraction of the whole CD4(+) T cell repertoire and can be isolated from the peripheral blood of humans according to their cytokine profile. Freshly purified interferon-gamma, IL4, and IL-10-producing allergen-specific CD4(+) T cells display characteristics of Th1, Th2, and TReg like cells, respectively. TReg cells consistently represent the dominant subset specific for common environmental allergens in healthy individuals. In contrast, there is a high frequency of allergen-specific IL-4-secreting T cells in allergic individuals. TReg cells use multiple suppressive mechanisms, IL-10 and TGF-beta as secreted cytokines and cytotoxic T lymphocyte antigen 4 and programmed death 1 as surface molecules. Healthy and allergic individuals exhibit all three allergen-specific subsets in different proportions, indicating that a change in the dominant subset may lead to allergy development or recovery. Blocking the suppressor activity of TReg cells or increasing Th2 cell frequency enhances allergen-specific Th2 cell activation ex vivo. These results indicate that the balance between allergen-specific TReg cells and Th2 cells may be decisive in the development of allergy.

The influence of a parasitical helminth infection with Heligmosomoides polygyrus (H. polygyrus) on peanut allergy has been previously examined in mice. The results from this research indicates that an infection of H. polygyrus, a natural mouse parasite, protects peanut sensitized mice against peanut allergy and that a chronic helminth infection can block the induction of allergen-specific IgE by influencing the behavior of the peanut antigen specific Th cells that are required for this response. The results also indicate that parasitical helminth-dependent protection against allergic disease involves immunoregulatory mechanisms that block production of allergen-specific IgE (J. Immunol. 2002 Sep. 15;169(6):3284-92).

Other research has shown that chitin, a surface component of parasites, which induces the production of chitinases in lower life forms during infections with parasites, ameliorated Th2 inflammation and airway hyperresponsiveness and thus may be an important mediator of asthma (Science. 2004 Jun 11;304(5677): 1678-82. Chitin is a surface component of parasites and insects, and chitinases are induced in lower life forms during infections with these agents. Although chitin itself does not exist in humans, chitinases are present in the human genome. Researchers have shown that acidic mammalian chitinase (AMCase) is induced via a Th2-specific, interleukin-13 (IL-13)-mediated pathway in epithelial cells and macrophages in an aeroallergen asthma model and expressed in exaggerated quantities in human asthma. AMCase neutralization ameliorated Th2 inflammation and airway hyperresponsiveness, in part by inhibiting IL-13 pathway activation and chemokine induction. AMCase may thus be an important mediator of IL-13-induced responses in Th2-dominated disorders such as asthma).

Mechanisms used by parasites to evade the host may include a number of different immunoregulatory mechanisms known in the art as immunosupression. Immunosuppression is the reduction of the host's immune response either to the parasite specifically or to foreign antigens in general. In other words, immunosuppression can be either specific, i.e., supression of only the host's immune response to the parasite or more general, i.e., involving host's immune response to various nonparasite antigens. A variety of mechanisms have been suggested to explain the immunosupression, such as: (1) the presence in the infected host of parasite or host substances that nonspecifically stimulate the growth of antibody-producing B cells, rather than stimulating the proliferation of specific antiparasite B-cells; (2) proliferation of suppressor T-cells and/or macrophages that inhibit the immune system by excretion of regulatory cytokines; and (3) production by the parasite of specific immune suppressor substances.

Helminths release a variety of molecules, known in the art as excretory and secretory products (ESP), into the host, which are believed to play a role in host immunosuppression. Although the composition of ESP is largely unknown, it is a source of components for the treatment of allergic disease because ESP is able to induce protection for the helminth from the host's immune response through immunosuppression, and, in doing so, inhibits the induction of allergen-specific IgE by influencing the behavior of the antigen specific Th cells that are required for this response. Stated another way, helminth-dependent protection against allergic disease involves immunoregulatory mechanisms caused by the production of or the introduction of the helminth of specific immune suppressor substances, which blocks and/or regulates production of allergen-specific IgE.

The present invention provides novel methods and compositions for treatment of allergies mediated by an abnormal Th2 immune response. The novel methods of the invention comprise administration of a helminth compound to a non-natural host for treatment of the symptoms associated with allergies.

### SUMMARY OF THE INVENTION

The present invention relates to compositions and methods of treating disease states that are marked by abnormal IgE immune responses to benign antigens, including an inappropriate Th2 cell production. Specifically, the invention relates to administration of helminth compounds to reduce the excessive IgE immune response in a host. The methods and compositions of the invention may be used to treat allergies, including but not limited to food allergies, allergies caused by pollen, pet dander, dust mites as well as asthma.

In an embodiment of the present invention, a pathogen-free non-human colonizing helminth compound is administered to a host in an amount sufficient to establish a transitory parasitic helminth infection and or to simulate a parasitic helminth infection. Such compounds include, but are not limited to, the use of isolated helminths, helminth extracts and ESPs. The use of such compounds is believed to have an immunosuppressive effect against benign antigens and/or to stimulate a regulatory immune response characterized by the production of at least one of T helper cells 2 (Th2), T regulatory helper cells (TReg) and certain cytokines, including, but not limited to, interleukin 10 (IL-10). Such compounds may be used as a therapy or prophylaxis of allergy and other IgE-mediated disorders, which are marked by an inappropriate IgE immune response including, but not limited to, an aberrant and or enhanced IgE antibody production to benign antigens.

A method of the invention comprises the administration of helminth compounds in a frequency and amount sufficient to reduce, eliminate or ameliorate the inappropriate immune response in an asthmatic and or allergic individual. An embodiment of this invention is generally directed to diseases and IgE-mediated disorders, including asthma, allergies, hypersensitivity and anaphylactic reactions. More specifically, an embodiment of the present invention is directed toward the treatment of certain CFA's.

An embodiment of the present invention relates to compositions comprising a helminth compound. Such helminth compounds, include but are not limited to, a pathogen-free non-human colonizing helminth such as a live adult helminth, ground adult helminth, adult helminth extract, adult helminth ESP, live helminth larvae, ground helminth larvae, helminth larvae extract, helminth larvae ESP, live helminth eggs, ground helminth eggs, helminth eggs extract, and helminth eggs ESP. The helminth compound may comprise a helminth selected from the group of helminths that do not naturally colonize humans and are otherwise non-pathogenic to humans, but through a repeated transitory infection in the gastrointestinal mucosa or the simulation of the same, offer an immunological benefit to an asthmatic, allergic and or hypersensitive individual. The invention further relates to a pharmaceutical composition comprising a helminth compound in a pharmaceutically acceptable carrier.

The helminth compound may simulate a parasitical infection in the human, and, in doing so, stimulate the immune system in a way in which it may protect allergic humans from the inappropriate immune response associated with allergies and asthma. The helminth compound is made from the group of helminths that colonize other animals, but not humans, and have no associated pathology or reduced pathology in humans. The helminth compound derived from these groups may establish only a transient infection in the human or may simulate the same using ESP, and, in doing so, stimulate the immune system in a way in which it may protect allergic humans from the inappropriate immune response associated with allergies and asthma. This stimulation may be maintained by repeated administration of the helminth compound, i.e., repeating the transient infection or simulation of a parasitic helminth infection with the helminth compound.

In one embodiment of the invention, the helminth to be used is Haemonchus contortus (H. contortus), or ESP cultured there from, which may, as further described below, as a third stage juvenile larvae, locate itself temporarily in the stomach mucosa of humans who ingest it or, when ESP, to simulate a parasitic helminth infection. H. contortus is a nematode that infects small ruminants. It releases a variety of ESP into the host and, although the composition of ESP is largely unknown, it is able to induce protection from the natural host's immune response to eliminate it (A. Yatsuda. Comprehensive analysis of the secreted proteins of the parasite H. contortus reveals extensive sequence variation and differential immune recognition). In the natural host, sheep, H. contortus has been shown to induce a regulatory immune response characterized by the production certain Th2-skewing cytokines, including IL-10. IL-10 mRNA expression by abomasal lymph node (ALN) lymphocytes from H. contortus infected sheep was determined by gene specific, reverse transcriptase (RT) polymerase chain reaction (PCR). ALN lymphocytes from infected lambs were isolated by histopaque density gradients, plated in standard culture media and stimulated with conconavalin A for 16 hrs. RNA was extracted from these lymphocytes and submitted to RT-PCR analysis. Amplified products of the expected size (nucleotide base pairs) on agarose gels were visualized by ethidium bromide staining and ultraviolet illumination.

In additional embodiments of the invention, the helminth compound may comprise or be derived from the group of helminths from the families of Ostertagia, Trichostrongylus, Trichostrongylus, Bunostomum, Nematodiriasis, Oesophagostomum, Trichuriasis, Chabertia, or any other suitable helminth.

The present invention provides a method of treating, or prophylaxis, of allergic and other IgE-mediated disorders, including, but not limited to, asthma, allergies, specifically, CFA's, hypersensitivity and anaphylactic reactions, which are marked by an inappropriate IgE immune response including an abnormal or enhanced IgE antibody production to benign antigens. The method of the invention comprises the administration of a non-human colonizing helminth compound, one or more times, in an amount sufficient to establish a repeated transitory gastrointestinal infection and thereby stimulating a regulatory immune response characterized by the production of Th2, TReg and certain cytokines, including, but not limited to IL-10. Such treatment is designed to eliminate or ameliorate the inappropriate immune response in an asthmatic, allergic and or hypersensitive individual.

In addition, the invention relates to a method of producing and or manufacturing a helminth compound comprising isolating a helminth from the stool of a prepatory animal, cleaning the helminth, maintaining the helminth in a specific pathogen-free environment, and formulating a pharmaceutical composition and a pharmaceutically acceptable carrier.

In addition, the invention relates to a method of producing and or manufacturing a helminth ESP comprising cultivated the ESP from an adult helminth, helminth larvae, and or helminth eggs, cleaning the helmith ESP, maintaining the helminth ESP in a specific pathogen-free environment, and formulating a pharmaceutical composition and a pharmaceutically acceptable carrier.

An advantage of the present invention is the creation of a pharmaceutical composition comprised of a pathogen-free non-human colonizing helminth compound, which will have an immunosuppressive effect against benign antigens in an allergic individual.

### DETAILED DESCRIPTION OF THE PREFERRED EMBODIMENTS

Elevations of anti-inflammatory cytokines, such as, but not limited to, interleukin-10 (IL-10) that occur during long-term parasitic infections have been shown to be inversely correlated with allergic disease, such as asthma and allergy. Specifically, helminths incite an intestinal Th2 response, which can cause worm expulsion or limit the magnitude of infection. Employing immunosuppressive defenses, many helminth species survive for years within the gut, binary tree or mesenteric veins making thousands of eggs daily. Thus, these worms and/or their ova release molecules that reside the intestinal mucosal surface for years, inciting Th2-type inflammation. Infestation with helminths, which induce Th2-type inflammation, can decrease excessive immune response to unrelated allergens because they may cause the secretion of Th2, TReg and certain cytokines, including, but not limited to IL-10. Thus, the induction of a robust anti-inflammatory regulatory network by the persistent immune challenge created by a parasitic offers an explanation for the observed inverse association of many parasitic infections with allergic disease. Stated another way, the failure to be infected with parasite may predispose an individual to allergic disease, such as asthma and allergies.

The present invention relates to compositions and methods that may be used to treat allergies, including but not limited to food allergies, allergies caused by pollen, pet dander, dust mites as well as asthma. The invention relates to administration of helminth compounds to reduce the excessive IgE immune response in a human. Specifically, a transitory infection with the certain non-pathogenic helminths (i.e., helminths incapable of establishing a host/parasite relationship or incapable of reaching reproductive adulthood) of the GI tract or the simulation of a parasitic helminth infection with ESP offers a means of preventing or treating IgE-mediated disorders, including asthma, CFA's, hypersensitivity and anaphylactic reactions according to the invention. The initial or primary helminth infection is by a helminth compound that is only capable of making a transitory infection in humans or, with respect to ESP, simulating the same. In doing so, the immune response is the same as or similar to that of the immune response to a pathogenic parasite. In the case where the helminth compound is a live helminth, because humans are not the helminth compound's natural host, the parasite is unable to establish more than a transitory infection, dies, and is expelled from the body. In order to simulate a chronic parasitical infection, the administration of the non-human colonizing helminth compound is repeated until the disease symptoms abate.

The administration of such compounds results in a blocking of the induction of an allergen-specific IgE response and/or stimulating a regulatory immune response characterized by the production of T helper cells 2 (Th2), T regulatory helper cells (TReg) and certain cytokines, including, but not limited to interleukin 10 (IL-10). Such administration of helminth compounds can be used as a therapy, or prophylaxis, of allergy and other IgE-mediated disorders, which are marked by an inappropriate IgE immune response including, but not limited to an aberrant and or enhanced IgE antibody production to benign antigens.

Thus, the method of the invention comprises the administration of a helminth compound in a frequency and amount sufficient to eliminate, ameliorate or reduce the inappropriate immune response in an asthmatic and or allergic individual. This invention is generally directed to IgE-mediated disorders, including asthma, allergies, hypersensitivity and anaphylactic reactions. More specifically, an embodiment of the present invention is directed toward the treatment of CFA's. While the present invention discloses specific information about the treatment of CFA, the disclosure is in no way limiting to CFA's.

The present invention is based upon the discovery that diseases that involve hypersensitivity and anaphylactic reactions, which are marked by an excessive IgE immune response including an aberrant and or enhanced IgE antibody production to benign antigens, are treatable by the administration of a helminth compound preparation that will provide a method of creating an immune environment that is conducive to eliminating, ameliorating or reducing IgE-mediated disorders, including asthma, food allergies, hypersensitivity and anaphylactic reactions or vaccinating an individual against IgE-mediated disorders, including asthma, allergies, specifically, CFA's, hypersensitivity and anaphylactic reactions.

In one embodiment of the invention, the helminth compound is from a helminth that naturally colonizes in sheep. Such parasites are species specific, i.e., such parasites only affect ruminants, such as sheep, and cannot be passed to humans or other monogastric animals. When introduced into a human host, the helminth is unable to reproduce or migrate from one host to another.

In an embodiment of the invention, the helminth H. contortus is utilized. In one embodiment of the invention, the third stage larvae (L3), is administered to a host in need of treatment. H. contortus has a very short life cycle in sheep and is located in the stomach glands (Scientific name: Haemonchus contortus; Common name: Barber pole worm; Class: Secernentea; Subclass: Rhabdita; Order: Stongylida; Superfamily: Trichostrongyloidea; Family: Trichostrongylidea). Natural hosts for H. contortus are sheep, goats, cattle, and wild ruminants. H. contortus is more prevalent in warm moist regions than in cold, dry ones. No intermediate host is required during its life cycle. Adult male and female worms live in the abomasum (or true stomach) of ruminant animals. The female deposits 5,000 to 10,000 eggs per day which pass out of the host with the feces. First stage juveniles hatch from the eggs. First and second stage juveniles feed on bacteria. Third stage juveniles retain the second stage cuticle as a sheath. Third stage juveniles do not feed and are infective for the vertebrate host. The ruminant becomes infected while grazing by eating the third-stage juveniles. Exsheathment occurs in the rumen, anterior to the abomasum, and the young worms pass into the abomasum where they burrow into the mucosa. Here they undergo another molt, and the fourth-stage juveniles come back into the paramucosal lumen of the abomasum. They begin to feed and undergo another molt before reaching adulthood. Mating of adults occurs and egg production commences. The eggs hatch in soil or water and develop directly to infective third-stage juveniles. Enormous numbers of juveniles may accumulate on heavily grazed pastures. The family contains many genera and species.

The males are 10 to 20 mm and the females 18 to 30 mm long. The white uteri and ovaries winding around the red blood-filled intestine give a twisted or barberpole appearance. The small buccal capsule bears a curved dorsal tooth. There are two prominent lateral spike-like cervical papillae near the junction of the first and second quarters of the esophagus. The male bursa has long lateral lobes and slender rays with a flap-like dorsal lobe located asymmetrically near the base of the left lateral lobe. The spicules are 450 to 500 um long, each with a terminal barb; the gubernaculum is navicular. Usually, the vulva is covered by an anterior thumb-like flap which may be reduced to a mere knob in some individuals. The oval eggs, are somewhat yellowish, and are 70 to 85 um long by 41 to 44 um wide in the early stages of cleavage when laid.

In yet another embodiment of the invention, several other ruminant helminths from the families of Ostertagia and Trichostrongylus, which are found in the stomachs of sheep, may be used as helminth compounds. Specifically, several species of Trichostrongylus are found in the small intestines. Sheep hookworms (Bunostomum) are found in the small intestine. Roundworms (Nematodiriasis) are also found in the small intestine. Another internal parasite of sheep found in the small intestine is the nodular worm (Oesophagostomum). Whipworms (Trichuriasis) and large mouth bowel worms (Chabertia) are found in the large intestines.

In one embodiment of the invention, compositions comprising microscopically small live H. contortus exsheathed third stage juvenile larvae (L3) will be administered to an individual suffering from allergies or having asthma. It is believed that such compounds will provide the most profound Th2 mucosal conditioning because of their ability to make a transient infection in the human stomach mucosa and their ability to release a variety of ESP, which in natural host (sheep and goats) is able to induce up to 90% protection from the host's immune response to eliminate it.

In one embodiment of the present invention, it is contemplated that L3 will establish an infection in humans, but that infection should be transient, to avoid complications that may arise from a prolonged infection. Transient H. contortus infection has been established in Mongolian gerbils, with the strongest establishment in immunocompromised Mongolian gerbils. The infection was expelled over time and fully mature (reproductively competent) worms were not observed. In some experiments with immunosuppressed mice, H. contortus did transiently infect the immunosuppressed mice. Mice and Mongolian gerbils, like humans, are monogastric.

The helmith compounds of the invention may be produced using a variety of different methods. In one method, sheep are used to produce such compounds. It is anticipated that the sheep, as the preparatory animal, will be specific pathogen free (SPF) sheep and raised in a pathogen-free environment according to methods known in the art (and as described below) and infected with H. contortus. The sheep will be tested to ensure the absence of human bacterial, mycobacterial, and viral pathogens and may need to be treated with: i) immunosuppressive glucocorticoids or azathioprine; ii) agents that impede Th2 effects like anti-histamines, anti-cytokines, or recombinant cytokines; and agents that influence intestinal moffiity like anti-cholinergics or opiates. Sheep will be tested to ensure a genetic background that renders them resistant to scrapie (a spongiform encephalopathy). Specific pathogen free (SPF) means sheep which are free of certain specific diseases and other disease causing microbes or pathogens. SPF sheep are bred, born, reared, maintained in environments which prevent exposure to or transmission of pathogens.

SPF sheep shall be free from virus, pneumonia, infectious atrophic rhinitis, external parasites, vibrio coli dysentery, and any other disease or condition spread by direct contact. SPF sheep shall be derived from a licensed laboratory for the production of SPF sheep and only as follows: (a) by the conventional hysterectomy procedure; (b) by laparotomy or caesarian section, in which: (1) there is accepted practice of strict surgical asepsis; and (2) this sheep's first breath is taken in an area protected from non-SPF animals. The latter may be accomplished by: (a) passing the sheep from the uterus into a separate room with a separate air supply; (b) passing the sheep from the uterus through a disinfectant water lock into a receptacle; or (c) the closed method which is the removal of the uterus and placing same in a sterile receptacle, where the sheep is removed.

A licensed laboratory for the production of SPF sheep shall be inspected and approved periodically by the national SPF advisory committee. A SPF sheep herd shall be a closed sheep herd that originates solely from a licensed laboratory. Any additions to this SPF herd must be laboratory sheep from a licensed laboratory. The exchange of male stock between SPF sheep herds may be permitted, if completed under the supervision of a licensed veterinarian. All health and disease inspections shall be made by a licensed accredited veterinarian. If, after this inspection, gross evidence of disease is established, further laboratory analysis shall be made. External parasites in a SPF herd will be cause for suspending SPF status until the parasitic condition is eliminated. The SPF sheep herd shall be validated as brucellosis-free, according to existing state-federal brucellosis regulations. All inspections, reports, tests, vaccinations, surgical procedures, accreditation, reaccreditation, or any other methods or procedures necessary to accredit, and maintain accreditation of, SPF sheep herds, shall be done by a veterinarian, or by some other appropriate individual who shall not have any financial interest in the sheep herd involved; except when special permission to perform any of these acts is granted by the livestock sanitary commissioner or his authorized representative.

In an effort to keep sheep free of pathogens, sheep housed in SPF-managed areas are maintained in rooms that are specifically designated for SPF sheep. Special caging and cleaning procedures shall be implemented in these areas and research personnel shall follow special procedures (including, but not limited to the procedures set forth below) to minimize the potential of pathogen transmission from a contaminated area or animal. For example, shoe covers, gown, and gloves must be worn while in the rooms designated as SPF. In rooms containing SPF sheep, a head bonnet and an additional pair of shoe covers shall be worn and removed before exiting the room. Gowns, exam gloves, and shoe covers provided in each SPF room/area should only be worn in that particular room/area.

Infections will be initiated with L3 infective larvae that are orally inoculated into SPF sheep. L3 will associate with mucosa of the abomasums (or true stomach), where they undergo development. L4 develop by 4 days post-infections and non-reproductively active adult worms developed by 7 days post-infection. Adult H. contortus are located on the mucosa of the abomasums and females begin producing eggs by about 18 days post-infection, approximately 5,000 to 10,000 eggs per day, which pass out of the sheep with the feces. The SPF sheep's diet may be altered to reduce coarse fiber content and oral purgative to induce defecation. The oval eggs are 70 to 85 um long by 41 to 44 um wide and in the early stages of cleavage when laid. They are somewhat yellowish. The stool is collected and enzymatically digested to free the H. contortus eggs. The feces (fecal pellets) are collected for culture of H. contortus eggs to infective L3. To produce infective L3, fecal pellets containing eggs are dispersed, mixed with vermiculite and cultured under conditions known in the art. Specifically, the eggs are then isolated from liquefied stool by flotation on density gradients, screen filtration, Visser filtration, or centrifugal elutriation. Processed to render them bacteria and virus free, the eggs then require a maturation phase and are incubated under optimal conditions to mature the embryo, or hatch the egg and provide L3 forms. Specifically, first stage juveniles (L1) hatch from the eggs and feed on fecal bacteria in a pathogen-free environment, grows and molts to a second stage juvenile (L2). The L2 continues to feed, grows and molts into a L3. The L3 in invective to the next host (ruminant) when ingested. The L3 maintains the molted cutile (sheath) of the L2 which envelops the L3 infective larva. L3 are motile, and under defined conditions, they migrate out of the fecal culture. Migration out of the fecal culture allows the isolation of the L3 by sedimentation in a Baermann apparatus, free from the bulk of the fecal and vermiculite culture material. Isolated L3 are further purified to render them free from any potential pathogens (bacteria, virus, and fungi) by using the following steps. The second stage cuticle that ensheaths the L3 protects these larvae from noxious chemicals. The ensheathed L3 are cleansed with the strong denaturing detergent sodium dodecyl sulfate (1%) which will destroy pathogens that might contaminate the preparation. L3 are then separated from remaining debris by density gradient separation. Isolated L3 remain ensheathed during this process. This preparation of L3 can be stored for months in sterile water at 10 degrees C.

Prior to infection, L3 are exsheathed in order to enhance L3's ability to establish a transitory infection by bubbling CO₂ into sterile water, by a method disclosed herein, or any other method known in art. Exsheathed L3 are then treated with a solution of sodium hypochlorite (0.1% W:V) for 10 minutes, which is a stringent antimicrobial and antiviral treatment, but is not toxic to L3. The last step may not be required if the detergent treatment is adequate. Exsheathed L3 prepared in this manner are pelleted by centrifugation, washed in sterile water, pelleted and washed again. Specifically, exsheath L3 in a solution of hypochlorite prior to inoculation into mice by using the following protocol.
1. Gently resuspend L3 by mixing the stock preparation and add 1 ml of L3 to a 15 ml polycarbonate orange cap conical tube. Heat L3 (2,500) at 37° C. for 5 min.
2. Add 10 ul of bleach (6% hypochlorite) to 1 ml of L3 (ca. 1% bleach, 0.06% hypochlorite). Gently mix immediately and thoroughly by pipeting.
3. Incubate for 20 minutes at room temperature. Monitor the effect on low power objective of an inverted microscope. Check at 10, 15 and 20 min.
4. Empty sheaths should be obvious and abundant in the solution after 20 min.
5. Add 9 mls of ice cold, sterile, double distilled water. Visually inspect the solution and identify individual L3 that are suspended. This step will aid assessment of successful pelleting of L3 by centrifugation in the next step.
6. Centrifuge in table top swinging bucket at 300×g for 3 min. Use medium braking.
7. Inspect the solution for a pellet (all L3 should be in a small loose pellet) and for possible L3 that might remain suspended. Additional centrifugation might be required if all L3 are not pelleted. This assessment needs to be made by eye because microscopic examination will cause turbulence and resuspension of L3.
8. Carefully aspirate 9 mls of solution, with suction applied at the top, leaving 1 ml buffer zone above the L3. This process will prevent turbulence from resuspending L3 at the bottom. Follow this process closely by eye.
9. Repeat steps 5-8 three times to wash the L3 pellet. The concentration of bleach will have been diluted to 1:100,000, which is below levels considered acceptable in drinking water (e.g. 1:25,000).
10. Once the sample is centrifuged and aspirated to 1 ml of L3 solution, resuspend the L3 pellet. This will require gentle and repeated pipeting (1 ml pipettor). Observe the pellet during the process to confirm suspension of single L3 without L3 clumps. Otherwise pipet until this presentation is achieved.

Infection competency will be determined in mice that are treated with 0.02% hydrocortisone in feed. Infection efficiency will be assessed seven days post-infection and efficiencies of 20% or higher are expected. Exsheathed L3 (do not need 100% exsheathment) should be at the original concentration. L3 should be maintained at room temperature until use. Each L3 preparation will be rigorously tested to confirm absence of bacterial and viral pathogens using standard microbiological techniques. For example, the following may be used: bioburden, mycoplasma, host-specific viruses, sterility and endotoxin. This final preparation of L3 is used to initiate infection. The helminth compound will be formulated for oral dosage at an acceptable pH with conventional filters, carriers and excipients know in the art or presented in water.

L3 will not feed and are ready to establish a transitory infection in individuals in need of treatment. It will be refrigerated at between 45 degrees and 50 degrees Fahrenheit. The males are 10 to 20 mm and the females 18 to 30 mm long. The white uteri and ovaries winding around the red blood-filled intestine give a twisted or barberpole appearance. The small buccal capsule bears a curved dorsal tooth. There are two prominent lateral spike-like cervical papillae near the junction of the first and second quarters of the esophagus. The male bursa has long lateral lobes and slender rays with a flap-like dorsal lobe located asymmetrically near the base of the left lateral lobe. The spicules are 450 to 500 um long, each with a terminal barb; the gubemaculum is navicular. Usually, an anterior thumb-like flap that may be reduced to a mere knob in some individuals covers the vulva.

The pharmaceutical compositions of the invention comprise L3 and a pharmaceutically acceptable carrier. In an embodiment of the invention the composition is designed for oral administration. Orally ingested, the L3 will survive in body after being ingested and will establish an infecfion in the individual, which will be transient thereby avoiding the complications that may arise from a prolonged infection. In yet another embodiment of the invention, the helminth compounds may be formulated for injection into the host.

In order to avoid any egg production in the individual, the invention anticipates that L3 composition comprises only males, or alternatively, is enriched for males. Males and females may be separated by flotation on density gradients, screen filtration, Visser filtration, or centrifugal elutriation. It is anticipated that an amount ranging from about 1,000 to about 100,000 L3 will deposit themselves and establish a transitory infection in the mucosa of the stomach using the curved dorsal tooth. Because they are in the wrong (unnatural) host, L3 will die before they reach the fourth stage of development and are capable of reproduction. Dead, L3 will loosen from the stomach mucosa and will be eliminated completely during bowel movement. L3 will not be visible in the stool. It is anticipated that individuals with asthma and or allergies will repetitively drink doses of L3 at intervals of 7 to 14 days in order to simulate an infestation of the parasites.

The helminth compound of the invention will be formulated for oral dosage, at an acceptable pH with conventional fillers, carriers, and excipients known in the art or presented in water. Such compositions may be presented for use in conventional manner with the aid of any necessary pharmaceutical carriers or excipients. The amount of helminth administered to the individual in need thereof is an amount sufficient to prevent, reduce the severity of, or treat the disease, which may vary depending upon the individual or disease being treated or prevented, but is anticipated to range from about 1,000 to about 100,000 L3.

In order to show the efficacy of the present invention, the assessment of allergic symptoms and anaphylactic response may be monitored. There are several well-established disease activity indices that monitor clinical parameters in an asthmatic and or allergic individuals, including evaluating the allergic response following oral challenge with the CFA, as well as laboratory and histological criteria.

The Th2 and TReg response is determined by assaying serum cytokine and immunoglobulin concentrations, cytokines and immunoglobulins, IL-4, IL-5, IL-10 and IL-13 and IgE and IgG1 characterizing a Th2 and TReg. Using these indices, disease activity in the individual is monitored and evaluated. In the absence of disease symptoms, L3 treatment will be discontinued. Upon the return of disease symptoms, L3 treatment will resume. Patients are monitored for four months for anemia, adult worms or ova by fecal flotation, gastritis, diarrhea, constipation. If there is evidence of a persistent helminth infection, patient will be treated with one of the following anthelmintics: Albenza (albendazole), Ergamisol (levamisole hydrochloride), Stromectol (ivermection)

In another preferred embodiment of the present invention, it is contemplated that the helminth compound will be ESP and will, by simulating the establishment of a parasitical helminth infection in humans, have an immunosuppressive effect against benign antigens by blocking the induction of allergen-specific IgE or stimulating a regulatory immune response characterized by the production of Th2, T TReg and certain cytokines, including, but not limited to IL-10. The oral administration in mice of secreted proteins in soluble form cultured from Nippostrongylus brasiliensis (N. brasiliensis), a natural parasite of mice, causes a Th2 response (A. Balic, et al. Eur. J. Immunol. 2004. 34: 3047-3059).

For preparation of ESP, H. contortus may be established in sheep as described above. The sheep will be sacrificed and approximately 10,000 adult H. contortus worms will be harvested. Specifically, adult H. contortus worms will be collected at day 6 post infection and cultured for 7 days in RPMI 1640 with 100 U/ml penicillin, 100 lg/ml streptomycin and 1% glucose. Supernatants will be collected at 48-hour intervals from days 1 through 7, pooled and concentrated to 1 mg/ml. Potential endotoxin contamination is neutralized by pre-incubation with 20 lg/ml polymyxin B sulfate (Sigma) at 37C for 30 minutes (A. Balic, et al. Eur. J. Immunol. 2004. 34: 3047-3059). The pharmaceutical compositions of the invention comprise ESP and a pharmaceutically acceptable carrier. In an embodiment of the invention the composition is designed for oral administration. In yet another embodiment of the invention, the helminth compounds may be formulated for injection into the host.

### EXAMPLE 1

Heligmosomoides polygyrus (H. polygyrus) is parasite of rodents and mice infected with H. polygyrus have been shown to be protected against peanut allergy (J. Immunol. 2002 Sep. 15;169(6):3284-92).

Infecting mice with H. polygyrus stimulates the gut-associated immune system and results in typical Th2 immune responses (F, Finkelman, Annu Rev Immunol 1997;15:505-533). H. contortus is not a parasite of rodents, i.e., its natural hosts are not mice and have no associate pathology in mice. However, infecting mice with H. contortus also results in a Th2 skewed immune response. Specifically, seven Balb/c female mice (Groups 2 and 3) were fed approximately 500 H. contortus L3 by oral gavage in accordance with the frequency set forth in Table 1. Three mice (Group 1) were used as the control and not fed H. contortus L3. The serum from all groups was collected and the total IgE level of the serum was determined by ELISA using two anti-mouse IgE monoclonal antibodies. In doing so, total IgE level of the serum was determined to be statically significantly greater in the mice fed H. contortus L3 (Groups 2 and 3) when compared with the control (Group 1). The total IgE level of the serum was observed in the quantities set forth in Table 2.

These results indicate that H. contortus is capable of stimulating a Th2 cytokine response in an unnatural host, i.e., mice, and may possess helminth-dependent immunoregulatory mechanisms in an unnatural host which would block production of allergen-specific IgE (J. Immunol. 2002 Sep. 15; 169(6):3284-92).

TABLE 1
Dosing Schedule of Mice Treated with H. contortus L3

| | | Week 1 | Week 2 | Week 3 | Week 4 |
|---|---|---|---|---|---|
| Group | # of | (Day | (Day | (Day | (Day |
| # | Animals | 1-7) | 8-14) | 15-21) | 22-28) |
| 1 | 3 | — | — | — | — |
| 2 | 4 | D1; D4; D6 | D8; D11; D13 | — | — |
| 3 | 3 | D1; D4; D6 | D8 | D15 | D22 |

TABLE 2
Total IgE Levels in Serum from Mice
Treated with H. contortus L3

| Group # | Animal # | Total Serum IgE (ng/ml) |
|---|---|---|
| 1 | 1 | 3.4 ± 0.8 |
| | 2 | 5.2 ± 0.0 |
| | 3 | 3.8 ± 0.3 |
| 2 | 4 | 8.3 ± 0.9 |
| | 5 | 11.0 ± 2.2 |
| | 6 | 10.1 ± 0.3 |
| | 7 | 5.2 ± 0.6 |
| 3 | 8 | 13.3 ± 0.3 |
| | 9 | 16.6 ± 0.3 |
| | 10 | 21.1 ± 2.5 |

The entire disclosure of each of the cited literature references is incorporated herein by reference thereto.

It should be understood that the foregoing detailed description is provided for clarity only and is merely exemplary. The spirit and scope of the present invention are not limited to the above example, but are encompassed by the claims.

What is claimed is:

1. A method for producing a pharmaceutical composition comprising a third stage juvenile larvae helminth preparation, comprising the steps of:
(1) raising a preparatory animal in a pathogen-free environment;
(2) isolating the third stage juvenile larvae helminth from said preparatory animal to form a third stage larvae parasite isolate; and
(3) mixing the parasite isolate from step (2) with a pharmaceutically acceptable carrier.

2. The method of claim 1, wherein the step of isolating a helminth comprises obtaining a stool from said preparatory animal, and isolating the third stage juvenile larvae from said stool.

3. The method of claim 1, wherein the preparatory animal is raised in a specific human pathogen-free environment.

4. The method of claim 1 further including the step of confirming the absence of bacterial and viral pathogens in the third stage juvenile larvae.

5. The method of claim 1 wherein the isolated third stage larvae are ensheathed and comprising the step of removing the sheaths from at least one of said larvae.

6. The method of claim 5 wherein at least 50% of the sheaths are removed.

7. The method of claim 5 wherein the exsheathed larvae are treated with at least one of an antimicrobial and an antiviral solution.

8. The method of claim 1 wherein the third stage juvenile larvae helminth mature from Haemonchus contortus eggs.

9. A method of preparing a pathogen-free exsheathed third stage juvenile larvae comprising the steps of:
(1) isolating an ensheathed third stage juvenile larvae
(2) washing the ensheathed larvae with a denaturing detergent
(3) removing sheaths from at least one of the third stage juvenile larvae
(4) isolating the larvae

10. The method of claim 9 wherein the sheaths are removed by bubbling CO₂ into a water solution containing the ensheathed larvae.

11. The method of claim 9 further comprising the step of treating the exsheathed larvae with at least one of an antimicrobial and an antiviral solution.

12. The method of claim 11 wherein the at least one of an antimicrobial and an antiviral solution is a solution of sodium hypochlorite.

13. The method of claim 9 further comprising the step of confirming the absence of bacterial and viral pathogens in the third stage juvenile larvae.

14. The method of claim 9 wherein at least 50% of the sheaths are removed from the ensheathed larvae.

15. The method of claim 9 further comprising the step of separating the exsheathed and ensheathed larvae.

16. The method of claim 9 wherein the ensheathed third stage juvenile larvae matures from Haemonchus contortus eggs.

17. A method for producing a pharmaceutical composition comprising a helminth parasite preparation from the Class Secementea, comprising the steps of:
(1) raising a preparatory animal in a specific human pathogen-free environment;
(2) isolating a Class Secernentea helminth parasite from said preparatory animal to form a helminth parasite isolate; and
(3) mixing the parasite isolate from step (2) with a pharmaceutically acceptable carrier.

18. The method of claim 17 wherein Class Secernentea helminth parasite is Haemonchus contortus.

19. A method of treating a disorder characterized by an increase in the level of IgE comprising administering to a mammal in need thereof, a therapeutically effective amount of a helminth compound.

20. The method of claim 19 wherein said disease is asthma.

21. The method of claim 19 wherein said disease is an allergy.

22. The method of claim 21 wherein said allergy is a common food allergy.

23. The method of claim 19 wherein the helminth compound is selected from the families consisting of Ostertagia, Trichostrongylus, Trichostrongylus, Bunostomum, Nematodiriasis, Oesophagostomum, Trichuriasis and Chabertia.

24. The method of claim 23 wherein the helminth compound is Haemonchus contortus.

25. The method of claim 19 wherein the helminth compound is selected from the group consisting of live adult helminth, ground adult helminth, adult helminth extract, adult helminth ESP, live helminth larvae, ground helminth larvae, helminth larvae extract, helminth larvae ESP, live helminth eggs, ground helminth eggs, helminth eggs extract, and helminth eggs ESP.

26. The method of claim 19 wherein the helminth compound is third stage juvenile larvae.
